(19) United States
(12) Patent Application Publication
(10) Pub. No.: US 2022/0054412 A1
(43) Pub. Date: Feb. 24, 2022

(54) TOPICAL FORMULATION AND METHODS FOR NEUROPATHY RELIEF

(71) Applicant: Jason Pool, Horseshoe Bay, TX (US)

(72) Inventor: Jason Pool, Horseshoe Bay, TX (US)

(21) Appl. No.: 17/338,278

(22) Filed: Jun. 3, 2021

Related U.S. Application Data
(60) Provisional application No. 63/067,982, filed on Aug. 20, 2020.

(51) Int. Cl. A61K 9/06; A61K 9/00; A61K 31/167; A61K 31/135; A61K 31/5415; A61K 31/4168; A61K 31/4422; A61K 31/197

(52) U.S. Cl. CPC A61K 9/06; A61K 9/0014; A61K 31/167; A61K 31/197; A61K 31/5415; A61K 31/4168; A61K 31/4422; A61K 31/135

(57) ABSTRACT

Disclosed herein is a formulation for relief of symptoms associated with neuropathy, such as pain, burning, itching, or tingling. The formulation comprises ketamine HCL, gabapentin, piroxicam, clonidine HCL, nifedipine, and lidocaine HCL in a cream base for topical application for relief of neuropathy symptoms. Disclosed herein is a method of making a formulation for relief of symptoms associated with neuropathy, such as pain, burning, itching, or tingling, the formulation comprising ketamine HCL, gabapentin, piroxicam, clonidine HCL, nifedipine, and lidocaine HCL in a cream base suitable for topical application. Disclosed herein is a method of using a formulation for relief of symptoms associated with neuropathy, such as pain, burning, itching, or tingling, the formulation comprising ketamine HCL, gabapentin, piroxicam, clonidine HCL, nifedipine, and lidocaine HCL in a cream base for topical application to relieve symptoms of neuropathy.

### CROSS REFERENCE TO RELATED APPLICATIONS

This application claims the benefit of U.S. Provisional Application No. 63/067,982 filed Aug. 20, 2020, which is incorporated by reference into this utility patent application.

### STATEMENT REGARDING FEDERALLY SPONSORED RESEARCH OR DEVELOPMENT

Not applicable.

### TECHNICAL FIELD OF INVENTION

This invention relates to the field of treatment and relief of neuropathy symptoms.

### BACKGROUND OF THE INVENTION

Neuropathy is damage, disease, or dysfunction of one or more nerves, especially of the peripheral nervous system, with symptoms that may include pain, burning, itching, or tingling in the affected area. One or more nerves in an area may be affected and more than one area may be affected, for example, both hands and/or both feet. Peripheral neuropathy is a result of damage to the nerves outside of the brain and spinal column. Neuropathy can result from traumatic injury, viral or bacterial infections, toxin exposure, inherited disorders and metabolic disorders. Common types of peripheral neuropathy include diabetic neuropathy, chemo-therapy induced neuropathy, HIV-related neuropathy, post-herpetic neuralgia, and trigeminal neuralgia.

The symptoms associated with neuropathy can range from mildly uncomfortable to severely painful or disruptive. Symptoms may have an acute onset or be associated with a chronic condition. The symptoms may progress or increase over time and the neuropathy may become a chronic condition with long-term consequences.

Treatments have been used for neuropathy, often including oral medications. Traditional oral pain medications, such as opioids, are often ineffective for alleviating neuropathic pain. Other types of oral medications, such as calcium channel subunits, tricyclic antidepressants, and selective serotonin receptor inhibitors, have been used with varying degrees of success; however, many of these oral medications have unwanted side-effects. There remains a need for an effective treatment for neuropathic symptoms without unwanted side-effects. This need may be met with topical formulations that provide relief from symptoms while avoiding the side effects seen with oral medications.

### SUMMARY OF THE INVENTION

Disclosed herein is a neuropathy relief formulation to treat symptoms associated with neuropathy, including pain, burning, itching, or tingling of an affected area. The disclosed is a formulation for topical application for relief of neuropathy symptoms. Disclosed herein is a method of making a neuropathy relief formulation topical preparation for relief of symptoms associated with neuropathy. Also disclosed is a method of using the neuropathy relief formulation for treatment of the symptoms of neuropathy, including pain, burning, itching, or tingling.

Disclosed herein is a method of making a formulation, the method comprising the steps of: providing therapeutic ingredients comprising ketamine HCL, gabapentin, piroxicam, clonidine HCL, nifedipine, and lidocaine HCL; preparing a substantially homogenous powder mixture of the therapeutic ingredients; providing one or more pharmaceutically suitable carrier suitable for topical application of therapeutic ingredients; combining the substantially homogenous powdered mixture of the therapeutic ingredients with the one or more pharmaceutically suitable carrier to produce a combined substantially homogenous powdered mixture and pharmaceutically suitable carrier; processing the combined substantially homogenous powdered mixture and pharmaceutically suitable carrier in an ointment mill to produce a substantially homogenous topical formulation having therapeutic concentrations of ketamine HCL, gabapentin, piroxicam, clonidine HCL, nifedipine, and lidocaine HCL.

The disclosed substantially homogenous topical formulation has ketamine HCL 1% to 20% w/w, gabapentin 1% to 20% w/w, piroxicam 0.1% to 20%, clonidine HCL 0.01% to 10% w/w, nifedipine 0.01-20% w/w, and lidocaine 1-20% w/w in a pharmaceutically suitable carrier. The substantially homogenous topical formulation may have 10% w/w ketamine HCL, 6% w/w gabapentin, 1% w/w piroxicam, 0.2% w/w clonidine HCL, 2% w/w nifedipine, and 5% w/w lidocaine HCL.

The therapeutic ingredients of the disclosed substantially homogenous topical formulation may further comprise 0.5%-20% w/w baclofen. The therapeutic ingredients may further comprise 0.5%-20% w/w amitriptyline. The therapeutic ingredients may further comprise 0.5%-20% w/w cyclobenzaprine. The substantially homogenous topical formulation may have one or more of baclofen 2%-5% w/w, amitriptyline 2%-5% w/w, and cyclobenzaprine 2%-5% w/w.

In the disclosed substantially homogenous topical formulation, the one or more pharmaceutically suitable carrier comprises one or more of a humectant, penetrant, protectant, emollient, preservative, and a moisturizing ingredient suitable for topical delivery of pharmaceutical therapeutics. The substantially homogenous topical formulation is a treatment for peripheral neuropathy.

Disclosed herein is a topical formulation comprising: ketamine HCL, gabapentin, piroxicam, clonidine HCL, nifedipine, and lidocaine HCL in a pharmaceutically suitable carrier. The disclosed topical formulation has 2%-10% w/w ketamine HCL. The topical formulation has 2%-10% w/w gabapentin. The topical formulation has 0.5%-10% w/w piroxicam. The topical formulation has 0.1%-5% w/w clonidine HCL. The topical formulation hs 0.1%-10% w/w nifedipine. The topical formulation has 2%-10% w/w lidocaine.

In the disclosed topical formulation, the pharmaceutically suitable carrier has at least one emulsifying agent. The pharmaceutically suitable carrier has one or more of a humectant, penetrant, protectant, emollient, preservative, and moisturizing ingredient suitable for topical delivery of pharmaceutical therapeutics.

The disclosed topical formulation may further comprise one or more of 0.5%-20% w/w baclofen, 0.5%-20% w/w amitriptyline, and 0.5%-20% w/w cyclobenzaprine. The topical formulation is a treatment for peripheral neuropathy. The topical formulation may be applied topically to an affected area one, two, three or four times a day for treatment of peripheral neuropathy.

Disclosed herein a topical formulation comprising: 2%-10% w/w ketamine HCL; 2%-10% w/w gabapentin; 0.5%-10% w/w piroxicam; 0.1%-5% w/w clonidine HCL; 0.1%-10% w/w nifedipine; 2%-10% w/w lidocaine; and a pharmaceutically suitable carrier having at least one emulsifying agent, and one or more of a humectant, penetrant, protectant, emollient, preservative, and moisturizing ingredient suitable for topical delivery of pharmaceutical therapeutics. The topical formulation may have 10% w/w ketamine HCL, 6% w/w gabapentin, 1% w/w piroxicam, 0.2% w/w clonidine HCL, 2% w/w nifedipine, and 5% w/w lidocaine HCL.

The disclosed topical formulation may further comprise 0.5%-20% w/w baclofen. The topical formulation may further comprise 0.5%-20% w/w amitriptyline. The topical formulation may further comprise 0.5%-20% w/w cyclobenzaprine. The topical formulation is a treatment for peripheral neuropathy. The topical formulation may be applied topically to an affected area one, two, three or four times a day for treatment of peripheral neuropathy.

### DETAILED DESCRIPTION OF THE INVENTION

Disclosed herein is a topical formulation and methods for relief of symptoms of neuropathy. Disclosed herein is a method of making a formulation for topical application to relieve symptoms of neuropathy. Disclosed herein is a method of using a topical formulation for relief of symptoms of neuropathy.

In a preferred embodiment for topical application to the skin, the disclosed neuropathy relief formulation comprises a synergistic mixture of ketamine HCL, gabapentin, piroxicam, clonidine HCL, nifedipine, and lidocaine in a pharmaceutically suitable carrier for relief of symptoms of neuropathy.

The disclosed neuropathy relief formulation is useful for relieving pain, burning, itching, or tingling. or other symptoms associated with neuropathy. In a preferred formulation, the therapeutic agents are ketamine HCL, gabapentin, piroxicam, clonidine HCL, nifedipine, and lidocaine in pharmaceutically suitable carrier for topical application to an area experiencing symptoms of neuropathy. These therapeutic agents work synergistically to alleviate symptoms. The neuropathy relief formulation is typically applied topically to area three to four times daily for relief of pain, burning, itching, or tingling associated with peripheral neuropathy. The formulation may include one or more of baclofen, amitryptyline, and cyclobenzaprine.

Ketamine HCL is an anesthetic agent used as a sedative for diagnostic and surgical procedures that can also be used topically. Topically applied ketamine HCL can be used to treat acute neuropathic pain and other symptoms associated with neuropathy. Ketamine HCL can comprise 1%-20% of the disclosed formulation. The disclosed formulation may have 2% to 10% w/w ketamine HCL. The disclosed formulation may have 2% to 5% w/w ketamine HCL. The disclosed formulation may have 10% w/w ketamine HCL.

Gabapentin is an anti-epileptic drug that can be used to treat peripheral neuropathic pain. The medication works by decreasing the transmission of pain signals to the brain. Gabapentin can comprise 1%-20% of the disclosed formulation. The disclosed formulation may have 2% to 10% w/w gabapentin. The disclosed formulation may have 2% to 5% w/w gabapentin. The disclosed formulation may have 6% w/w gabapentin.

Piroxicam is a nonsteroidal anti-inflammatory drug (NSAID) that works by reducing the production of prostaglandins in the body that cause pain and inflammation. Piroxicam can comprise 0.1%-20% of the disclosed formulation. The disclosed formulation may have 0.5-10% w/w piroxicam. The disclosed formulation may have 1-5% w/w piroxicam. The disclosed formulation may have 1% w/w piroxicam.

Clonidine HCL can be used topically to improve penetration of the active ingredients in affected tissue. Clonidine HCL can comprise 0.01%-10% of the disclosed formulation. The disclosed formulation may have 0.1-% to 5% w/w clonidine HCL. The disclosed formulation may have 0.2-% to 3% w/w clonidine HCL. The disclosed formulation may have 0.3% w/w clonidine HCL. The disclosed formulation may have 0.2% w/w clonidine HCL.

Nifedipine can be used topically to reduce vasoconstriction in affected tissue. Nifedipine can comprise 0.01%-20% of the disclosed formulation. The disclosed formulation may have 0.1% to 10% w/w nifedipine. The disclosed formulation may have 0.5% to 5% w/w nifedipine. The disclosed formulation may have 2% w/w nifedipine.

Lidocaine is a local anesthetic that can be used to relieve pain and itching. The medication works by causing temporary numbness or loss of feeling in the area where applied. Lidocaine can comprise 1%-20% of the disclosed formulation. The disclosed formulation may have 2% to 10% lidocaine HCL. The disclosed formulation may have 3% to 5% lidocaine HCL. The disclosed formulation may have 5% w/w lidocaine HCL.

The therapeutic agents are combined with one or more pharmaceutically suitable carrier compatible with hydrophobic and hydrophilic therapeutic actives to produce a substantially smooth cream or ointment formulation for topical administration. Pharmaceutically suitable carriers will utilize one or more emulsifying agent to stably disperse lipid and aqueous components and is used in combination with pharmaceutically acceptable humectant, penetrant, protectant, emollient, surfactant and/or moisturizing ingredients suitable for topical delivery of pharmaceutical therapeutic agents. Pharmaceutically suitable carrier components may include one or more of alcohols, diols, fatty acids, fatty acid esters, fatty alcohols, oils, glycols, polyols, behenyl alcohol, butylene glycol, caprylic acids, cetyl alcohol, cetearyl alcohol, glycerides, glycerin, hexylene glycol, methylpropane diol, mineral oils, petrolatum, propylene glycol, sodium laureth sulfate, steric acid, stearyl alcohol, urea, vegetable oils. Additional excipient ingredients may be included in the pharmaceutically suitable carrier, such as stabilizing, solubilizing, and preservative ingredients.

Disclosed is a formulation having a synergistic combination of ketamine HCL 1% to 20% w/w, gabapentin 1% to 20% w/w, piroxicam 0.1% to 20%, clonidine HCL 0.01% to 10% w/w, nifedipine 0.01-20% w/w, and lidocaine 1-20% w/w in a pharmaceutically suitable carrier, such as an emulsifying base, for topical application. In one preferred embodiment, the formulation has 10% ketamine HCL, 6% gabapentin, 1% piroxicam, 0.2% clonidine HCL, 2% nifedipine, and 5% lidocaine HCL in a pharmaceutically suitable carrier for topical application of the therapeutic agents.

The formulation may include one or more of baclofen, amitryptyline, cyclobenzaprine.

Baclofen is a muscle relaxer and antispasmodic agent that can be applied topically to relieve localized chronic pain. The disclosed formulation may include 0.5%-20% w/w baclofen. The disclosed formulation may include 1.0%-10% w/w baclofen. The disclosed formulation may include 2.0%-5% w/w baclofen.

Amitriptyline can inhibit histamine receptors. Topically applied amitryptyline affects neurotransmitters associated with itching. The disclosed formulation may include 0.5%-20%. The disclosed formulation may include 3.0%-10% w/w amitryptyline. The disclosed formulation may include 2%-5% w/w amitryptyline.

Cyclobenzaprine works to reduce muscle spasms. Topically applied, cyclobenzaprine blocks pain signals to the brain. The disclosed formulation may include 0.5%-20% w/w cyclobenzaprine. The disclosed formulation may include 1%-10% w/w cyclobenzaprine. The disclosed formulation may include 2%-5% w/w cyclobenzaprine.

### Exemplary Preparation of Neuropathy Relief Formulation

To prepare the neuropathy relief formulation, effective amounts of ketamine, gabapentin, piroxicam, clonidine, nifedipine, and lidocaine are combined using geometric dilution to produce a substantially uniform mixture. In geometric dilution, components having the smallest quantities are added to an equal quantity of the component having the greatest quantity and thoroughly combined. The process is continued until all of the components have been combined into a substantially homogeneous powder mixture.

The geometric dilution mixing of the powdered components is accomplished via pharmaceutically accepted methods including trituration, spatulation, sifting, blade grinding or tumbling of the powders. Trituration with a mortar and pestle can be used to grind the active ingredients to a substantially uniform powder. A powder spatula and tile can be used to grind the active ingredients to a substantially uniform powder. Sifting with a fine mesh sieve can be used to produce a substantially uniform powder mixture of the active ingredients. A blade grinder can be used to grind the active ingredients to a substantially uniform powder. A manual or mechanical tumbler can be used to produce a substantially uniform powder mixture of the active ingredients. A substantially uniform powder is also referenced as a substantially homogeneous powder. By substantially uniform or substantially homogeneous, the composition or powder would be a substantially uniform consistency with 85%-100% fully mixed to meet that substantial uniformity.

The powdered active ingredients are combined with a pharmaceutically suitable carrier compatible with hydrophobic and hydrophilic therapeutic actives and stirred until the mixture is substantially homogenous. An emulsifying cream base utilizes one or more emulsifying agent to stably disperse lipid and aqueous components and is used in combination with pharmaceutically acceptable humectant, penetrant, protectant, emollient, preservative, and/or moisturizing ingredients that are suitable for topical delivery of pharmaceutical therapeutics. Pharmaceutically suitable carriers may be an individual carrier component, combinations of individual carrier components, or one or more commercially available carrier combinations. An example of a commercially available pharmaceutical carrier combination is an emulsifying cream base, Humco PENcream™, however other types of emulsifying bases, cream bases, emulsifying cream bases, or combinations of emulsifying cream or ointment -type bases that are compatible with the therapeutic components may also be used.

Once the therapeutic ingredients and the emulsifying base are substantially uniformly combined to a substantially homogeneously cream-type mixture, the resulting cream-type mixture is processed by a mechanical incorporation procedure to produce a substantially smooth formulation with therapeutic agents substantially uniformly distributed therein. The resulting cream is processed with an ointment mill or a blade mixer to produce a substantially uniformly smooth final composition. The homogenous mixture is processed through an ointment mill to reduce the particle sizes of the active ingredients and produce a substantially uniformly smooth final composition. A roller-type ointment mill uses high shear force to reduce and disperse the particles in the cream base to produce a substantially homogenous composition with a smooth texture. An exemplary milling method processes the cream through the ointment mill at least two times. The roller mill gaps are set to balance throughput of the material through the mill with the desired dispersion of the particles in the composition. An exemplary setting for the roller mill is a gap setting of 2 in the front and 2 in the back. When the neuropathy relief formulation is fully milled, the finished product is dispensed in 1, 2, or 3 ounce metal tubes, pursuant to prescription requirements.

### Exemplary Embodiment of the Neuropathy Relief Formulation

The neuropathy relief formulation has ketamine HCL 1% to 20% w/w, gabapentin 1% to 20% w/w, piroxicam 0.1% to 20%, clonidine HCL 0.01% to 10% w/w, nifedipine 0.01-20% w/w, and lidocaine 1-20% w/w in a pharmaceutically suitable carrier, such as an emulsifying base, for topical application. In one preferred embodiment, the formulation has 10% ketamine HCL, 6% gabapentin, 1% piroxicam, 0.2% clonidine HCL, 2% nifedipine, and 5% lidocaine HCL in one or more pharmaceutically suitable carrier for topical application of the therapeutic agents.

In one embodiment, the neuro relief formulation has the active ingredients in the following concentrations: 10% ketamine HCL, 6% gabapentin, 1% piroxicam, 0.2% clonidine HCL, 2% nifedipine, and 5% lidocaine HCL in a pharmaceutically suitable carrier. An exemplary formulation is shown in Table 1 with the active ingredients combined with an emulsifying cream base suitable for topical drug application. The exemplary formulation is one example only with higher and lower concentrations of the therapeutic agents envisioned, depending on the treatment required.

TABLE 1

| | Amount | |
|---|---|---|
| Active Ingredient (Final | | |
| Concentration % w/w) | | |
| Ketamine HCL USP (10%) | 90 | grams |
| Gabapentin USP (6%) | 54 | grams |
| Piroxicam USP (1%) | 9 | grams |
| Clonidine HCL USP (0.2%) | 1.8 | grams |
| Nifedipine USP (2%) | 18 | grams |
| Lidocaine HCL USP (5%) | 45 | grams |
| | 217.8 | grams |
| | (total actives) | |
| Inactive Compounding Ingredients | | |
| Emulsifying Base | 682.2 | grams |

(add to active
ingredients to q.s.
to 900 grams)

To prepare 900 grams of the exemplary neuropathy relief formulation, 90 grams of ketamine, 54 grams of gabapentin, 9 grams of piroxicam, 1.8 grams of clonidine, 18 grams of nifedipine, and 45 grams of lidocaine are combined using geometric dilution to produce a substantially uniform mixture. Trituration with a mortar and pestle can be used to grind the active ingredients to a substantially uniform powder. A powder spatula and tile can be used to grind the active ingredients to a substantially uniform powder. A blade grinder can be used to grind the active ingredients to a substantially uniform powder. A manual or mechanical tumbler can be used to produce a substantially uniform powder mixture of the active ingredients.

The resulting substantially uniform powder of active ingredients is combined with one or more pharmaceutically suitable carrier compatible with hydrophobic and hydrophilic therapeutic agents and stirred until the mixture is substantially homogenous. In the exemplary process, the powdered active ingredients are combined with 682.2 grams of an emulsifying base and mixed until a substantially homogenous cream-type mixture results.

An emulsifying concentrate utilizes one or more emulsifying agent to stably disperse lipid and aqueous components and is used in combination with pharmaceutically acceptable humectant, penetrant, protectant, emollient, preservative, and/or moisturizing ingredients that are suitable for topical delivery of pharmaceutical therapeutics. Pharmaceutically suitable carriers may be an individual carrier component, combinations of individual carrier components, or one or more commercially available carrier combinations. An example of a commercially available pharmaceutical carrier combination is an emulsifying cream base, Humco PENcream™, however other types of emulsifying bases, cream bases, emulsifying cream bases, or combinations of emulsifying cream or ointment -type bases that are compatible with the therapeutic components may also be used.

Once the active ingredients and the emulsifying base are homogeneously combined, the resulting cream is processed by a mechanical incorporation procedure to produce a substantially smooth formulation with therapeutic agents substantially uniformly distributed therein. The resulting cream is processed with an ointment mill or a blade mixer to produce a substantially uniformly smooth final composition. The resulting cream is processed through an ointment mill to reduce the particle sizes of the active ingredients and produce a substantially uniformly smooth final composition. A roller-type ointment mill uses high shear force to reduce and disperse the particles in the cream base to produce a homogenous composition. An exemplary milling method processes the cream through the ointment mill at least two times. The roller mill gaps are set to balance throughput of the material through the mill with the desired dispersion of the particles in the composition. An exemplary setting for the roller mill is a gap setting of 2 in the front and 2 in the back. When the neuropathy relief formulation is fully milled, the finished product is dispensed in 1, 2, or 3 ounce metal tubes, pursuant to prescription requirements.

Embodiments of the neuropathy relief formulation within the scope of this disclosure may have concentrations of the therapeutic agents, ketamine HCL, gabapentin, piroxicam, clonidine HCL, nifedipine, and lidocaine HCL, that differ from the exemplary embodiment in Table 1. In embodiments having the therapeutic in concentrations that differ from the exemplary embodiment, the quantity of emulsifying base is adjusted proportionally in order to maintain the desired concentration of the therapeutic agents. Preparations of the neuropathy relief formulation with more or less than the concentration of therapeutic agents in the exemplary embodiment above will be made in the same manner. If concentrations of the therapeutic agents are increased, the amount of emollient cream base will be decreased proportionally to achieve the desired concentration of the therapeutic agents. If concentrations of the active ingredients decrease, the amount of emollient cream base will increase proportionally to achieve the desired concentration of the therapeutic agents.

The neuropathy relief formulation may have one or more of baclofen, amitryptyline, and cyclobenzaprine included in the therapeutic agents. In an embodiment where the formulation incorporates one or more of the optional therapeutic agents, preparation of the neuropathy relief formulation will be similar to the exemplary embodiment process above. In an embodiment of the formulation including baclofen, amitryptyline, cyclobenzaprine or combinations thereof, the amount of emulsifying base will be adjusted proportionally in order to maintain the desired concentration of the therapeutic agents.

### Exemplary Use of Neuropathy Relief Formulation

The neuropathy relief formulation is applied in an amount sufficient to cover the affected area of clean dry skin. The neuropathy relief formulation is rubbed into the affected area until no cream remains visible on the surface. The formulation can be applied one, two, three or four times per day as needed for relief of symptoms.

Significant benefits of the formulation have been seen when used for neuropathy symptoms, including, but not limited to, pain, itch, burning, and tingling. The combination of therapeutic agents in the neuropathy relief formulation has been found to work synergistically to alleviate symptoms more effectively than the individual therapeutics. The following example cases are from a few of the individuals who have been successfully treated with the claimed formulation

### EXAMPLE 1

Patient JP had experienced severe, debilitating neuropathy for six years. Numerous medications including methadone and high doses of gabapentin had been tried but were unsuccessful. JP was unable to sleep for more than an hour at a time due to severe foot pain. JP found that use of the disclosed formulation reduced the pain significantly and allowed him to sleep four to five hours at a time, and has allowed him to return to activities, such as hunting.

### EXAMPLE 2

Patient SK had suffered with constant pain and itching in the feet due to neuropathy. SK was prescribed the disclosed formulation and upon using the formulation, experienced a substantial reduction in the neuropathy pain. SK has been able to return to normal activities.

### EXAMPLE 3

Patient MT is a pharmacist and had experienced neuropathy in both feet of two years duration. The neuropathic pain limited the ability of MT to work as a pharmacist. MT was prescribed and began using the disclosed formulation, and the neuropathy symptoms have been substantially alleviated, such that MT has not only been able to return to work, but has been able to work extra hours.

1. A method of making a formulation, the method comprising the steps of:
providing therapeutic ingredients comprising ketamine HCL, gabapentin, piroxicam, clonidine HCL, nifedipine, and lidocaine HCL;
preparing a substantially homogenous powder mixture of said therapeutic ingredients;
providing one or more pharmaceutically suitable carrier suitable for topical application of therapeutic ingredients;
combining the substantially homogenous powdered mixture of the therapeutic ingredients with said one or more pharmaceutically suitable carrier to produce a combined substantially homogenous powdered mixture and pharmaceutically suitable carrier;
processing the combined substantially homogenous powdered mixture and pharmaceutically suitable carrier in an ointment mill to produce a substantially homogenous topical formulation having therapeutic concentrations of ketamine HCL, gabapentin, piroxicam, clonidine HCL, nifedipine, and lidocaine HCL.

2. The method of claim 1, wherein the substantially homogenous topical formulation has ketamine HCL 1% to 20% w/w, gabapentin 1% to 20% w/w, piroxicam 0.1% to 20%, clonidine HCL 0.01% to 10% w/w, nifedipine 0.01-20% w/w, and lidocaine 1-20% w/w in a pharmaceutically suitable carrier.

3. The method of claim 2, wherein the substantially homogenous topical formulation has 10% w/w ketamine HCL, 6% w/w gabapentin, 1% w/w piroxicam, 0.2% w/w clonidine HCL, 2% w/w nifedipine, and 5% w/w lidocaine HCL.

4. The method of claim 1, wherein the therapeutic ingredients further comprise 0.5%-20% w/w baclofen.

5. The method of claim 1 wherein the therapeutic ingredients further comprise 0.5%-20% w/w amitriptyline.

6. The method of claim 1, wherein the therapeutic ingredients further comprise 0.5%-20% w/w cyclobenzaprine.

7. The method of claim 2, wherein the substantially homogenous topical formulation has one or more of baclofen 2%-5% w/w, amitriptyline 2%-5% w/w, and cyclobenzaprine 2%-5% w/w.

8. The method of claim 1, wherein said one or more pharmaceutically suitable carrier comprises one or more of a humectant, penetrant, protectant, emollient, preservative, and a moisturizing ingredient suitable for topical delivery of pharmaceutical therapeutics.

9. The method of claim 1, wherein the substantially homogenous topical formulation is a treatment for peripheral neuropathy.

10. A topical formulation comprising: ketamine HCL, gabapentin, piroxicam, clonidine HCL, nifedipine, and lidocaine HCL in a pharmaceutically suitable carrier.

11. The topical formulation of claim 10 having 2%-10% w/w ketamine HCL.

12. The topical formulation of claim 10 having 2%-10% w/w gabapentin.

13. The topical formulation of claim 10 having 0.5%-10% w/w piroxicam.

14. The topical formulation of claim 10 having 0.1%-5% w/w clonidine HCL.

15. The topical formulation of claim 10 having 0.1%-10% w/w nifedipine.

16. The topical formulation of claim 10 having 2%-10% w/w lidocaine.

17. The topical formulation of claim 10 wherein the pharmaceutically suitable carrier has at least one emulsifying agent.

18. The topical formulation of claim 17 wherein said pharmaceutically suitable carrier has one or more of a humectant, penetrant, protectant, emollient, preservative, and moisturizing ingredient suitable for topical delivery of pharmaceutical therapeutics.

19. The topical formulation of claim 10 further comprising one or more of 0.5%-20% w/w baclofen, 0.5%-20% w/w amitriptyline, and 0.5%-20% w/w cyclobenzaprine.

20. The topical formulation of claim 10 wherein the topical formulation is a treatment for peripheral neuropathy.

21. The topical formulation of claim 20 wherein the formulation is applied topically to an affected area one, two, three or four times a day for treatment of peripheral neuropathy.

22. A topical formulation comprising:
2%-10% w/w ketamine HCL
2%-10% w/w gabapentin
0.5%-10% w/w piroxicam
0.1%-5% w/w clonidine HCL
0.1%-10% w/w nifedipine
2%-10% w/w lidocaine; and
a pharmaceutically suitable carrier having at least one emulsifying agent, and one or more of a humectant, penetrant, protectant, emollient, preservative, and moisturizing ingredient suitable for topical delivery of pharmaceutical therapeutics.

23. The topical formulation of claim 22, having 10% w/w ketamine HCL, 6% w/w gabapentin, 1% w/w piroxicam, 0.2% w/w clonidine HCL, 2% w/w nifedipine, and 5% w/w lidocaine HCL.

24. The topical formulation of claim 22 further comprising 0.5%-20% w/w baclofen.

25. The topical formulation of claim 22 further comprising 0.5%-20% w/w amitriptyline.

26. The topical formulation of claim 22 further comprising 0.5%-20% w/w cyclobenzaprine.

27. The topical formulation of claim 22 wherein the topical formulation is a treatment for peripheral neuropathy.

28. The topical formulation of claim 27 wherein the formulation is applied topically to an affected area one, two, three or four times a day for treatment of peripheral neuropathy.
